(19) United States
(12) Patent Application Publication
(10) Pub. No.: US 2007/0078472 A1
(43) Pub. Date: Apr. 5, 2007

(54) Gajendra Safe Surgical Knife - AKA- GSS knife

(76) Inventor: Gajendra Singh, Iowa City, IA (US)

Correspondence Address: GAJENDRA SINGH, 1031 WESTSIDE DRIVE, IOWA CITY, IA, 52246, US

(21) Appl. No.: 11/163,065

(22) Filed: Oct. 4, 2005

(51) Int. Cl. A61B 17/32

(52) U.S. Cl. 606/167

(57) ABSTRACT

Gajendra safe surgical knife has a hexagonal shape, capacity of retraction and protraction of blade and able to lock temporarily and permanently. This surgical knife can be protracted with a push of the base of inner sheath and can be retracted by gently pressing the holding button. The holding button is a spring loaded small mechanism which holds automatically the inner sheath and blade within it with the forward movement of inner sheath. A pressure on the holding button releases the knife and it automatically retracts inside the knife handle. The knife can be locked temporarily by moving a small knob on the back of the handle, it prevents accidental protraction of the knife blade. Knife can be locked permanently by pulling the base of the handle backwards. Once it is locked permanently, it can't be released without breaking the handle completely.

### BACKGROUND OF THE INVENTION

The invention of this surgical knife is to make the surgical knife safest possible with greatest ease of handling and comfort. Recently there is much increase in communicable disease contract after sharp injury, especially during the surgical procedures and passing the sharps back and forth. The most significant of these infections are HIV and Hepatitis B & C. All of them have different level of morbidity and mortality.

Though there have been many attempts by different people to solve these problems. U.S. Pat. No. 5,330,492 describes a knife with extendable cover sheath. U.S. Pat. Nos. 5,779,724 and 5,423,843 describes a knife which can be extended and retracted. U.S. Pat. No. 5,250,063 shows a knife with extendable cover sheath and manually holding the retraction the blade. U.S. Pat. No. 5,116,351 describes a safety scalpel with a protective sheath. U.S. Pat. Nos. 5,071,426 and 5,139,507 describes a surgical scalpel with retractable blade guard. U.S. Pat. No. 4,663,846 describes a cutter having a retractable and removable blade. U.S. Pat. No. 3,906,626 describes a surgical scalpel with permanent sheathing of the blade. U.S. Pat. No. D359356 describes a design for retractable surgical knife.

To minimize the risk of sharp injury and exposure to severe communicable diseases and to resolve problems of many of the earlier invented surgical knives. Its mechanism is very simple, easy to use, comfortable, precise, and inexpensive. This surgical knife is designed for a very comfortable grip, which prevents rolling of the knife in hands or slipping out of fingers with great ease of working with a precise angle if desires.

This knife completely encloses the blade inside the sheath and it can be locked temporarily and permanently. All of the mechanisms are inside the sheath and no parts of the mechanism are coming out of the shaft to entangle with anything. The knife can be retracted and protracted with one hand technique. The design of the knife is very safe as it cannot be protracted in temporary or permanent locked position. The retraction and protraction mechanism are present at the base of the surgical knife and it can be operated with one hand, therefore it minimizes the risk of injury. This is disposable and single use knife and there is no need to clean or change the blade. This knife can be protracted and retracted with push of a single finger, therefore it can be kept on the surgical field and it is not necessary to return the knife back and forth to nurse.

### BRIEF SUMMARY OF THE INVENTION

The present invention provides a surgical knife with a capacity of protraction, automatic retraction, temporary and permanent locking mechanisms. The surgical knife blade is attached to a solid rod which is covered by the inner sheath and that is covered by outer sheath. The inner sheath and solid rod are attached with each other at the base and they move together. The inner sheath glides snuggly and smoothly inside the outer sheath. The outer sheath has a small rectangular holding button which clicks down in the hole of inner sheath, and holds automatically the inner sheath and knife in protracted position. By pressing the holding button the knife can be released and retracted automatically in the handle.

A small knob at the posterior surface of the handle works as a temporary lock in neutral position. In the temporary lock position the knife can not be moved in any direction either forward or backward. The knob slides in a specially designed groove on the surface of the inner groove. By moving the knob from side to side, the knife can be change to a position where it can either protract or lock permanently. The design of the groove on the inner sheath make it very safe that it can be locked in a temporary position when not in use and it prevent accidental protraction of the blade even if it is pushed from the back. The other great advantage of this device is that the position of the knob makes the movement of knife only in one direction, means either it can pull backward or push forward. The advantage of this mechanism is to prevent accidental protraction of the knife while trying to lock it permanently or prevent locking permanently while trying to protract the knife.

Another and very distinguishing advantage of this surgical knife is its hexagonal design. This design makes it very comfortable to hold and work and very precise angulations of the blade if required. The another advantage of this hexagonal design is that it minimize the rolling of the handle in hands and slipping out of the fingers which can be very dangerous around vital structures. Another advantage of this hexagonal design is that it prevents rolling of the inner sheath inside the outer sheath; this prevents the wobbling of the blade, and accidental release of the lock mechanism.

One another advantage of the this device is that the blade base glides in and out in a snuggly fit groove which prevents the side to side wobbling of the blade.

Another very important safety structure of this knife is the permanent lock, which are 2 pairs of the interlocking flaps and once locked cannot be released without breaking the surgical knife handle completely. Except the blade, spring and metal clips at the tip, the entire handle is made of plastic. This device is disposable so there is no need of cleaning of the sharps. The hexagonal design, simple working mechanism with minimum parts, light weight plastic build and locking mechanisms makes it very comfortable to work with, safe and precise instrument for surgery.

### BRIEF DESCRIPTION OF THE DRAWING

In the description of the drawings reference numbers and letters indicate the corresponding figure or parts of the device.

FIG. 1A shows the left side view of the Gajendra Safe Surgical Knife in retracted, temporarily locked and inoperative safe condition. Part 1 is outer sheath, Part 2 is inner sheath, part 3 is the holding button, part 4 is the knob and part 6 is the tapered tip.

FIG. 1B shows the left side view of the knife in operative, protracted condition. Part 3 in this figure is holding button in clicked in position and part 5 is a surgical blade.

FIG. 1C shows the left side view of the knife in inoperable, retracted and permanently locked position.

FIG. 2A shows the anterior view of the knife in protracted position. Part 1 is outer sheath, part 2 is inner sheath, part 3 is the holding button, Part 5 is the knife blade and Part 6 is the tapered tip.

FIG. 2B shows the posterior view of the knife in protracted position. Part 4 in this figure is knob, part 9 is side groove for movement of the knob.

FIG. 3A is the longitudinal cut section left side view of the surgical knife in retracted and temporarily locked position. In this figure part 2 is the inner sheath, part 3 is the holding button, part 4 is the knob, part 5 is the blade, part 6 is tapered tip, part 7 is surgical blade base, part 8 is inner most solid rod, part 10 is interlocking flaps attached to the outer sheath, part 11 is metal coiled spring in relaxed position, part 12 is the longitudinal groove for holding the knife blade base, part 13 is the interlocking flap attached to knife blade base, part 16 is ring holding the spring and attached to the outer sheath.

FIG. 3B is the longitudinal cut section left side view of the surgical knife in protracted and operating condition. In this figure part 3 is the holding button in clicked in position, part 5 is the knife blade in protracted position, part 11 is the metal coiled spring in stretched position.

FIG. 3C is the longitudinal cut section left side view of the surgical knife in retracted and permanently locked position. In this figure part 11 is metal coiled spring in compressed condition, part 10 and 13 are interlocked flaps.

FIG. 4A is the longitudinal cut section anterior view of the surgical knife in retracted and temporarily locked position. In this figure part 1 is the outer sheath, part 2 is the inner sheath, part 5 is the surgical blade, part 6 is the tapered tip, part 7 is the knife blade base, part 8 is the innermost solid rod, part 10 are the interlocking flaps attached to outer sheath, part 11 is the spring in normal relaxed position, part 13 are the interlocking flaps attached to blade base, part 14 are the metal clips at tip, part 16 is the ring holding the spring and attached to outer sheath.

FIG. 4B is the longitudinal cut section anterior view of the surgical knife in protracted and operating condition. In this figure part 11 is the spring in stretched condition.

FIG. 4C is the longitudinal cut section anterior view of the surgical knife in retracted and permanently locked position. In this figure part 11 is the spring in compressed position, part 12 is the longitudinal groove, part 10 and 13 are locked interlocking flaps.

FIG. 5A is the exploded view of the outer sheath in longitudinal cut section in left side view. In this figure part 3 is the holding button with a small spring inside, part 4 is the knob, part 10 is the interlocking flap attached to the wall, part 12 is the longitudinal groove, and part 16 is the ring attached to the side walls to hold the spring.

FIG. 5B is the exploded view of the outer sheath in longitudinal cut section in the anterior view. In this figure part 3 is the holding button, part 6 is the tapered tip, part 10 are the interlocking flaps attached to the side walls, part 12 is the longitudinal groove to hold the knife blade base, part 14 are the metal clips, part 16 is the ring to hold spring.

FIG. 5C is the exploded view of the outer sheath in longitudinal cut section in the posterior view. In this figure part 4 is the knob, part 6 is the tapered tip, part 9 is the side groove, part 14 are metal clips, part 16 is the ring to hold the spring.

FIG. 6A is the exploded view of the inner sheath in the anterior view from outside. In this figure part 17 is the longitudinal hole in the anterior part of the inner sheath to hold the holding button to keep the knife in protracted position.

FIG. 6B is the exploded view of the inner sheath in the posterior view from outside. In this figure part 18 is the longitudinal groove on the surface of inner sheath to facilitate the sliding movement of the knob to bring the knife in protracted position. Part 19 is the longitudinal groove on the outer surface of the inner sheath to facilitate the sliding movement of the knob to bring the knife in permanent locked position. Part 20 is the transverse groove on the outer surface of the inner sheath to hold the knob and facilitate its side to side movements. This is the neutral position of the knob which keeps the knife in temporary locked position.

FIG. 6C is the exploded view of the inner sheath in the side view from outside. In this figure part 21 is the partially cut away part of the side walls of the inner sheath to accommodate the interlocking flaps and attachment of the spring holding ring to the outer sheath.

FIG. 7A is the exploded left side view of the knife blade attached to the base and solid rod. In this figure part 5 is the surgical blade, part 7 is the blade base, part 8 is a solid rod and part 13 is the interlocking flaps attached to the blade base.

FIG. 7B is the exploded anterior view of the knife blade attached to the base and solid rod. In this figure part 5 is the surgical blade, part 7 is the blade base, part 8 is the solid rod, part 13 are interlocking flaps attached to the side of blade base.

FIG. 8A is the metal coil spring in side view.

FIG. 8B is the holding button from side view with a small spring in it.

FIG. 8C is the knob in lateral view.

FIG. 8D is the ring in the top view. In this figure part 1 are the side attachments which attach the ring to the inner surface of outer sheath, part 2 is the central part of the ring where the spring attaches, part 3 in this figure is a circular hole from which the solid rod smoothly glide backwards and forwards.

FIG. 8E is the side view of the ring.

FIG. 8F is showing the detailed, exploded, anterior view of the interlocking flaps. In this figure part 10 is the flap attached to the inner surface of outer sheath. Part 13 is the flap attached to the blade base, part X and Y are tapered portion of the flap from inside to facilitate the locking and prevent unlocking.

### DETAILED DESCRIPTION OF THE INVENTION

This surgical knife can be manufactured by conventional and standard materials. The blade of the surgical knife can be made by standard steel or carbon steel blades of various numbers, sizes, and shapes. The spring and metal clips can be made by steel and the rest of the components can be made by any strong plastic material.

The GSS knife is invented to minimize the problem of sharp injury and to overcome the drawbacks of the other different designs of surgical knives.

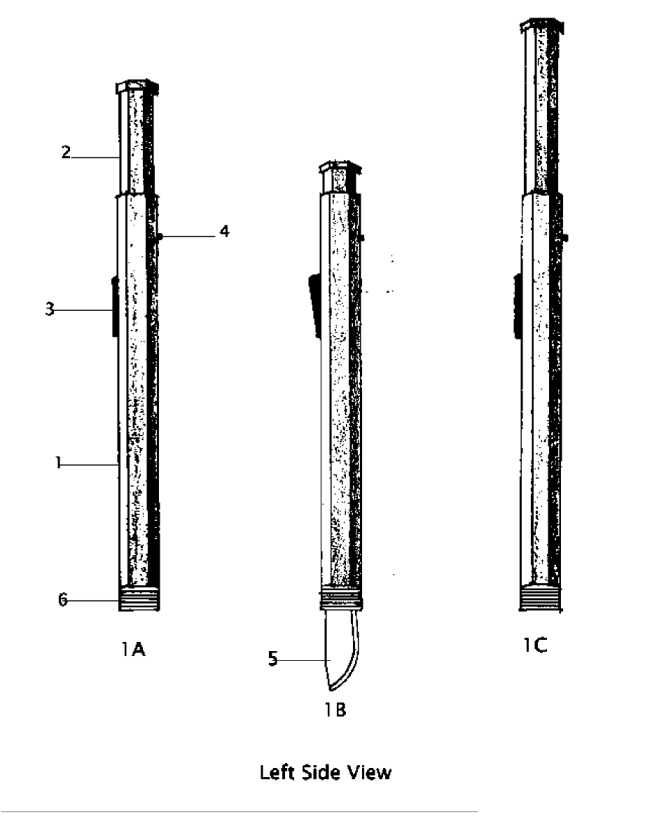

In the FIG. 1 the outside view of the GSS knife in depicted in three different drawings. FIG. 1A shows the knife in the normal retracted, and temporarily locked position. The knife will come in this position in package. In this position if the knife is pushed from back or bumped, it will not protract. This is safe and inoperative form of GSS knife.

To make the knife functional a small knob has to be moved to a side from its central position at the posterior surface of the outer sheath. This knob is shown as part 4 in FIG. 1A. By moving the knob from central position to left side the blade can be protracted by pushing the base of the knife or inner sheath. The inner sheath is shown as part 2 in FIG. 1A. The blade can be retracted by pressing the holding button gently on the anterior surface of the outer sheath. The holding button is shown as part 3 in FIG. 1A. The outer sheath is shown as part 1 in FIG. 1A.

In FIG. 1B the knife is shown in the protracted blade position.

FIG. 1C shows the knife in retracted and permanently locked position. By pulling on the base of the knife or inner sheath it can be locked permanently. To be able to lock the knife permanently the knob (part 4) has to be shifted from left side to right side. By pulling on the base and keeping the outer sheath immobilized the interlocking flaps come in approximation and then cross over to interlock. Once the flaps are locked, the design of the locking mechanism prevents unlocking. In the design of this device the blade itself folded by interlocking flaps and blade cannot be released unless the handle is broken completely. Even the breaking the base the handle will not release the blade and that makes the knife completely safe.

Another feature shown in the FIG. 1 is the hexagonal shape of the knife. Most of the designs of the surgical knives are flat i.e. U.S. Pat. Nos. 5,250,063, 5,116,351, 5,071,426, 3,906,626, or tubular like U.S. Pat. Nos. D 359,356, 5,779,724, 5,423,843, & 4,663,846. The flat and tubular design makes the knife uncomfortable to work with, if it has to be used for a long time. Some designs where the protective sheath covers the flat designs makes it more cumbersome to work with. The other disadvantages of the flat and tubular designs are slipping, rolling, and falling out of finger, and difficult to make straight incision and precise angulations, if working around the vital structures. These cumbersome designs and heavy weights cause early fatigue of the fingers and cause of inadvertent injuries either to the member of surgical team or to the patient. Metallic handles and flat designs sometimes hide these knives among other instruments and drapes. This increases the chance of accidental injuries.

To overcome with all these problems the GSS knife is designed in hexagonal shape and light weight plastic material. This design helps in perfect grip, straight incision lines and precise angulations if required. The handle of the knife will be color coded according to the size of the blade. The hexagonal shape and color of the handle will make it distinguishable among the other instrument and will further reduce the chances of injuries if the blade is left protracted.

Another advantage of the hexagonal design is that the inner sheath can't be rolled or move side to side inside the outer sheath. This feature helps in the smooth gliding of the inner sheath and prevents the rolling or wobbling of the blade with accidental release of the locking mechanism. In many of the other surgical knives if the locking mechanism is released accidentally, can extend the knife out side the protective sheath as it is under tension of a deforming force.

In this design it is a very remote possibility to release the permanent lock unless there is a manufacturing fault or handle is broken completely. The advantage of this device is that even after having the remote possibility, if the locks are released accidentally the blade will not come out of the handle. Even after accidental releasing of the permanent locks the blade will still stay inside the handle. All of these designs and working mechanisms makes a very remote possibility of accidental sharp injury by GSS knife, and that is possible only in the protracted blade if the knife is not used carefully.

Many of the other surgical knives are still reusable and blade has to be changed if required during surgery i.e. U.S. Pat. Nos. D359,356, 5,071,426, 5,250,063, 5,330,492, 5,779,724, 5,423,843. This increases the chance of accidental injuries. To overcome this problem this GSS knife is a single piece disposable knife. The blade cannot be separated from handle unless it is broken.

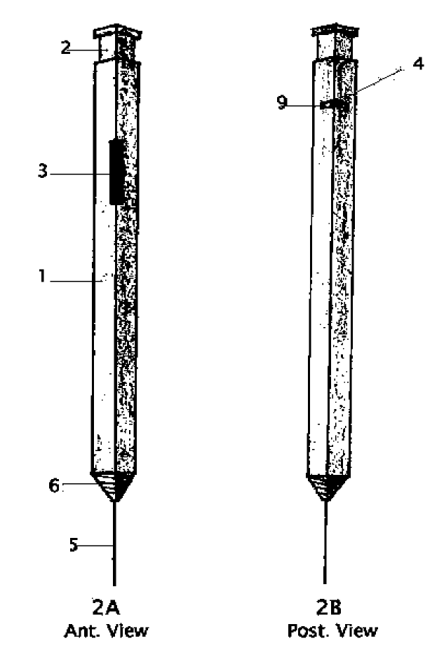

FIG. 2A shows the anterior and FIG. 2B shows the posterior view of the GSS knife In protracted position. In FIG. 2A part 3 is the holding button and part 5 is the blade and part 6 is the tapered tip. FIG. 2B part 4 is the knob and part 9 is the side groove.

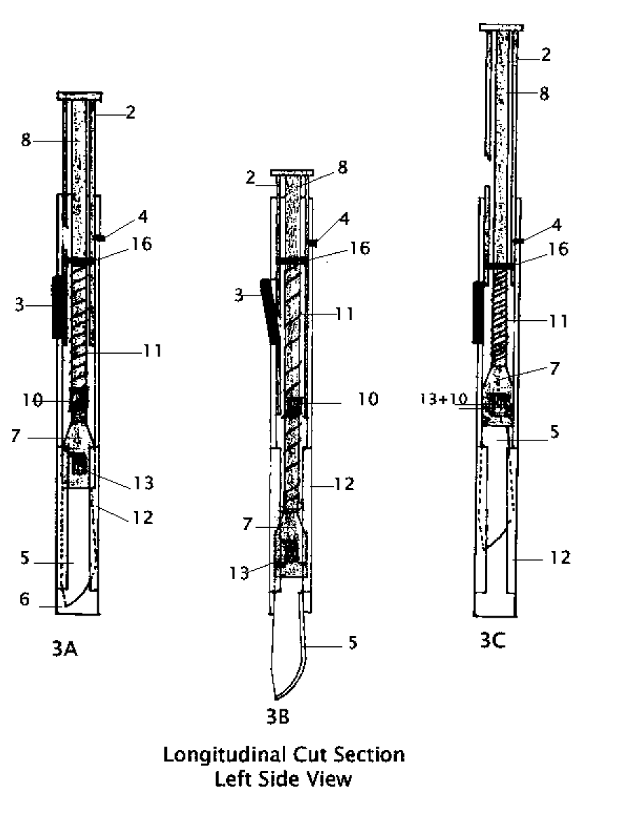

FIGS. 3A, 3B, 3C are drawings of the work mechanism longitudinal cut section of the knife in lateral view in three different positions. FIG. 3A is illustrating the knife in normal, retracted, inoperative and safe position. In this figure the knob (part 4) is holding the inner sheath (Part 2) In temporary locked position. FIG. 3B is showing the knife in protracted, operative condition. In this figure the holding button (Part 3) is clicked in and holding the inner sheath in protracted position and extending the blade (Part 5) outside of the sheath. In this position the metallic spring (Part 11 is in stretched condition. The blade can be released and retracted by gentle push on the holding button (part 3). By pushing the holding button the inner sheath will be released and the blade will be protracted inside.

The FIG. 3C is showing the knife in retracted and permanently locked position. In this figure the interlocking flaps (Part 10 and 13) are locked to keep the knife in permanently locked position. The metallic spring is in compressed position. In all these drawings part 16 is the ring holding the spring and attached to the outer sheath. This ring prevents the falling of all the internal structures from back of the handle.

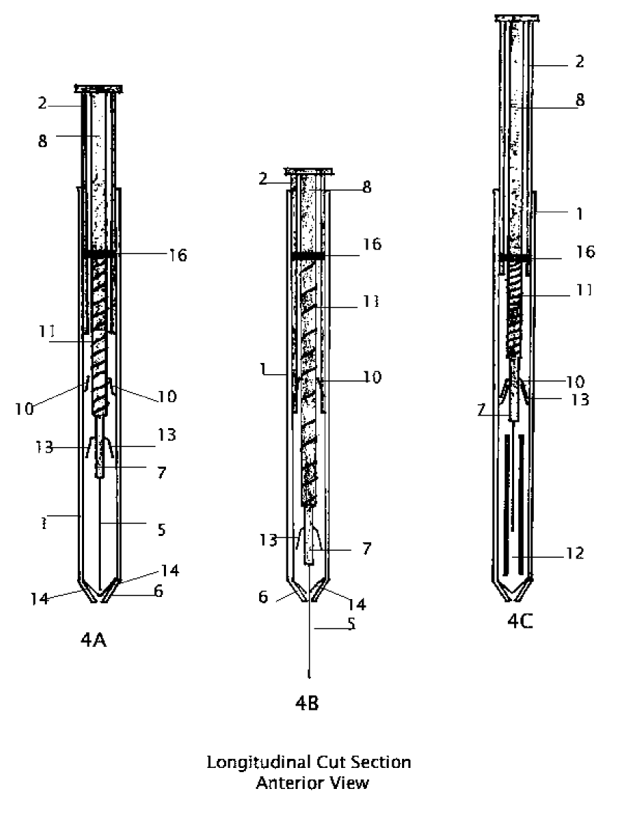

FIGS. 4A 4B and 4C shows the work mechanism in longitudinal cut section of the knife in anterior view. FIG. 4B shows the knife in protracted and operative condition. This figure illustrates the metal clips (part 14) at the tip, which prevents wobbling of the knife. In this figure relative positions of interlocking flaps, (Part 10 and 13) are shown. The metallic spring (Part 11) is in stretched position and in tension. When the holding button will be released this tension in the spring will bring the knife back in the handle.

FIG. 4C beautifully illustrates the permanent locking mechanism. The interlocking flaps (Part 10 and 13) are locked.

The blade is retracted in the sheath and it cannot be released without breaking the handle. In this figure Part 12 is the longitudinal groove in which base of the blade slides backwards and forwards. This grooves holds snuggly the base of blade and provides the stability to the blade during surgery and does not allow any wobbling.

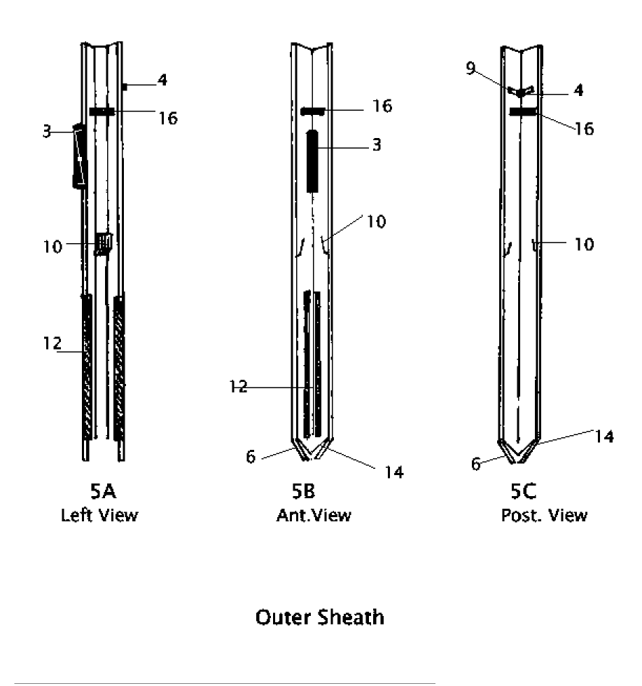

FIGS. 5A 5B and 5C are the illustrations of outer sheath in longitudinal cross section in left side, anterior and posterior view. FIG. 5A is the left side view in cross section. In this figure part 3 is the holding button with a small spring with in it. This spring causes clicking in of the holding button when the inner sheath is pushed down and holding button comes at the rectangular hole of inner sheath. Part 4 is the knob which moves from side to side to allow changing position from neutral to temporary and permanent locked position. Part 10 in this figure is the interlocking flap attached to the outer sheath wall and interlocks with the part 13. Part 12 in this figure is the longitudinal grove which snuggly holds the base of the and allow the smooth movements of the blade.

FIG. 5B is the cross section anterior view of the outer sheath. This shows the holding button (part 3) and longitudinal groove (part 12).

FIG. 5 C is the cross section posterior view of the outer sheath. This shows clearly the knob (part 4) which moves side to side in a small groove (part 9).

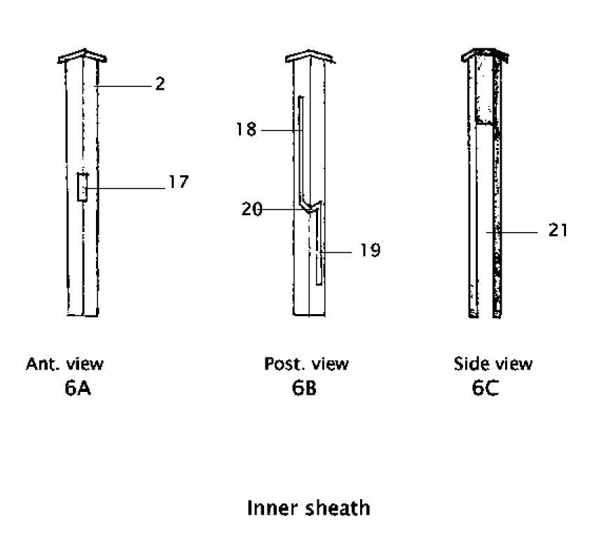

FIG. 6A is the anterior view of the inner sheath from out side. This figure shows the rectangular hole in the inner sheath. When the inner sheath is pushed down, this hole come in the place of the holding button in the outer sheath and this hole allows the clicking in of the holding button. This mechanism causes the blade to stay in the protracted position and makes it operable for surgery.

FIG. 6 B is the posterior view of the inner sheath. In this figure the groove (part 18, 19 and 20) on the posterior surface of the sheath are illustrated.

Part 20 the transverse groove allow the side to side movement of the knob. In the central position the knob is in temporary locked position and knife is inoperative and can't be moved in any direction. By moving the knob to the left side it makes the knife to be able to protract, as with the pushing of the base of the knife the knob will move in the groove 18. The knife can be moved in the forward direction only and on release of the holding button this will come back to the retracted position and repeatedly can be moved backward and forward position by pushing the base of the knife and releasing the holding button. But the design of the groove does not allow to go the knife in the permanent locked position. To make the knife to be able to lock permanently, the knob has to be moves from left side to the right side in the groove. In this position the knife can move only in backward direction only, not in forward direction.

Therefore in this position of the knob the knife can be locked permanently but can't be bring in the protracted position.

This special design prevents the risk of accidental injuries to minimum.

Because of this safety feature the knife can be kept on the field in retracted position and it is not necessary to return to the nurse, if there is rapid and frequent change of the instruments in any surgical procedure.

The surgeon can protract and retract the knife with a finger without any special efforts or time.

FIG. 6 C shows the inner sheath in the side view. In this figure the part 21 is the portion of the side walls which are cut away to give the space to accommodate the interlocking flaps (part 10) and attachments of the ring (part 16). The interlocking flaps and ring is attached to the inner surface of the outer sheath. The inner sheath is snuggly fit in the outer sheath and to accommodate these structures with smooth movements of the inner sheath, the side walls of the inner sheath are partially cut away.

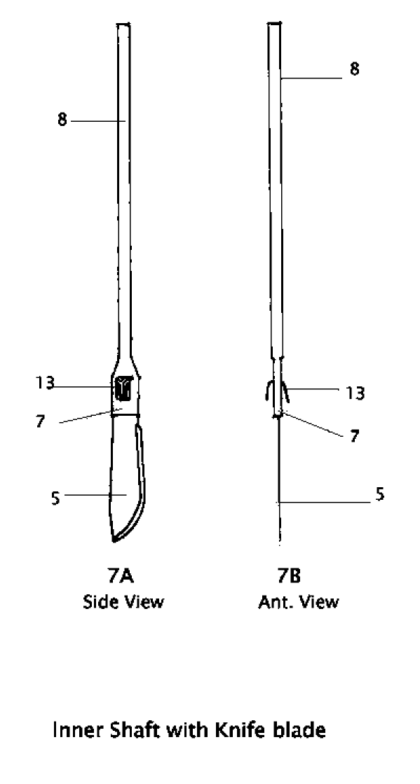

FIG. 7A shows illustrates the attachment of blade with the blade base and innermost solid rod in a single piece. The rod is attached to the base of the inner sheath and moves with the movement of the inner sheath. In this drawing the blade used is number 10 blade, the most commonly used blade in surgery. For practical purpose and manufacture any size of the blade can be used to make this knife. The base of the blade (part 7) is snuggly glides in the longitudinal groove (part 12). In this figure the part 13 illustrates the interlocking flaps attached to the blade base. The advantage of this is that the blade can only be released by breaking the knife handle completely, as after locking the knife permanently; the blade itself is locked in a secure position.

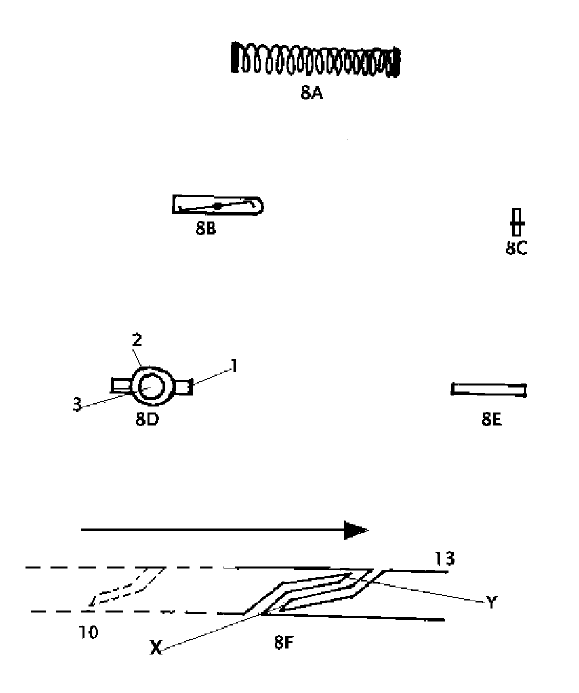

In FIG. 8A a compression metallic spring is illustrated. When the knife is in protracted position, the spring is under tension and with the release of the holding button, it bring the blade back in the sheath in the retracted position.

In FIG. 8 D the ring (part 16) is illustrated. In this figure the part 1 are a pair of side attachments which attaches to the walls of the outer sheath and provides the stability to the ring, and to the whole inner mechanism to function properly. Part 2 in this figure is the ring part where the metallic spring attaches. Part 3 is the circular inner hole which supports the innermost rod and allows the smooth gliding movements of the rod with the blade.

FIG. 8F illustrates the interlocking flaps 10 and 13. Part 10 is attached to the outer sheath and part 13 is attached to the blade base. To lock the knife permanently the base or inner sheath is pulled backward. This causes the specially designed flaps part 10 and 13 to cross over and then interlock. These flaps are made of plastic and the inner surface of the free end is tapered (part X & Y). This design helps to lock it and prevent unlocking.

Though, the present invention has been described in possible way of description, illustration and example, it will be understood that the present invention is not limited to the particular description and specific embodiments described but rather may comprise any combination of the above elements and variations. The disclosure of the details of the structure, function and mechanism are illustrative only, and changes can be made in detail, especially in matters of shape, size and arrangements of parts within the principle of the invention to the full extent indicated by the broad general meaning of the terms in the following claims.

What is claimed is:

1. A surgical knife which can be protracted and retracted with a temporary and permanent locking mechanism. The knife blade is attached to a handle which has one outer hexagonal sheath and one inner hexagonal sheath with an innermost solid rod. The rod is holding the knife and attached to the inner sheath and moves with the inner sheath. The inner sheath can move smoothly in and out of the outer sheath. A spring is loaded around the rod. This spring retracts the knife blade inside the handle when the holding button is pressed. By pressing the holding button, the inner sheath is released and the blade is retracted in the handle automatically. The tip of the handle is tapered side to side to give close and precise view of the incision line. Inside of the tip of the handle, there are 2 metal clips on sides to prevent the side to side movement of the blade. The blade slides in and out in a pair of snuggly fit grooves which prevent the wobbling of the blade. The small knob at the back side of the handle works as a temporary lock. By moving this knob from side to side the knife can be put in the position which makes it able to protract or lock permanently. The permanent locks are 2 pairs of interlocking flaps which once locked cannot be released.

2. The shape of the handle of surgical knife in claim 1 is hexagonal, which means it has 6 side surfaces to provide the best grip and prevents rotation and slipping of the knife handle.

3. The tip of the handle of the surgical knife in claim 1 is tapered from side to side to give the precise and closest view of the incision line.

4. The inner sheath of the surgical knife in the claim 1 is hexagonal and perfectly fit within the outer sheath to give the smooth gliding movements of the inner sheath inside the outer sheath.

5. The temporary lock mechanism of the surgical knife in the claim 1 is a small knob and a groove in the inner sheath. The knob slides in the groove on the surface of inner sheath. In the neutral position the knife is in the temporary lock and it can't either be protracted or locked permanently. By moving it side to side it can be put in the position to make it protract or lock permanently. The design of the grooves on the inner sheath doesn't allow protracting or locking permanently in the same knob position.

6. The permanent locks in the surgical knife in claim 1 are 2 pairs of interlocking plastic flaps which locks the knife blade itself. Once locked it can't be unlocked with out breaking the handle completely.

7. The holding button in the surgical knife in claim 1 is small plastic mechanism on the outer sheath with a small spring inside, which holds the inner sheath and blade in protracted position, when it is pushed downwards.

8. The metal clips in the surgical knife in claim 1 are a pair of flat metal clips at the tip of the handle. These clips facilitate the straight protraction of the blade with out hitting the wall of plastic holder and help to stabilize the knife blade.

9. The groove in the surgical knife in claim 1 are a pair of longitudinal groove at the front end of the handle inside the outer sheath. These grooves snuggly fits the base of the blade within it, and doesn't allow wobbling the knife blade.

10. The Spring in the surgical knife in claim 1 is a metal coil spring.

11. The inner sheath in the surgical knife in claim 1 has a rectangular hole in it anterior surface, which locks by the holding button when the sheath is pushed downwards.

12. The inner sheath in the surgical knife in claim 1 has specially designed groove for glide of the small knob to facilitate the knife in the protracted, temporary lock or permanent lock position.

13. The inner sheath in the surgical knife in claim 1 has it side walls cut away partially to accommodate the ring attachments and permanent interlocking flaps.

14. The interlocking flaps in the surgical knife in claim 1 are made of plastic, tapered at the tip from inside, to help locking and prevent unlocking.

15. The blade used in this design of the surgical knife in claim 1 is number 10 surgical blade, but any number of the blades can be used.

16. The Thickness and length of the surgical knife in claim 1 can be change according to the size of blade.

17. The only metal part in the surgical knife in claim 1 are knife blade, spring and metal clips. All of the other parts in the knife can be made by any kind of strong plastic material.

18. The surgical knife in claim is a single use knife and can be dispose after single use.
